(19) United States
(12) Patent Application Publication
(10) Pub. No.: US 2025/0329069 A1
(43) Pub. Date: Oct. 23, 2025

(54) METHOD OF TRAINING AN ARTIFICIAL NEURAL NETWORK FOR RECONSTRUCTING OPTOACOUSTIC AND ULTRASONIC IMAGES AND SYSTEM USING THE TRAINED ARTIFICIAL NEURAL NETWORK

(71) Applicants: Helmholtz Zentrum München Deutsches Forschungszentrum für Gesundheit und Umwelt (GmbH), Neuherberg (DE); iThera Medical GmbH, München (DE)

(72) Inventors: Dominik Jüstel, München (DE); Christoph Dehner, München (DE); Stefan Morscher, Schnaitsee (DE); Guillaume Zahnd, München (DE); Antonia Longo, München (DE)

(21) Appl. No.: 18/864,782

(22) PCT Filed: Jun. 1, 2023
(86) PCT No.: PCT/EP2023/064714

§ 371 Date: Nov. 11, 2024

(30) Foreign Application Priority Data
Jun. 3, 2022 (EP) 22177153.8

(51) Int. Cl. G06T 11/00; A61B 5/00; A61B 8/00

(52) U.S. Cl. CPC G06T 11/006; A61B 5/0095; A61B 8/5207; A61B 8/5261; A61B 8/5269; G06T 2210/41; G06T 2211/441

(57) ABSTRACT

The invention relates to a computer-implemented method and corresponding system for optoacoustic and ultrasonic imaging, a method for reconstructing optoacoustic and ultrasonic images and a method for training an artificial neural network provided therefor, the training method comprising: a) providing a model of the imaging apparatus, the model characterizing a relation between i) a spatial distribution of acoustic sources emitting and/or reflecting acoustic waves and ii) signals generated by detection elements of the imaging apparatus upon detecting the acoustic waves, b) providing several training signal sets, each training signal set comprising a plurality of training signals which were i) generated by the imaging apparatus upon imaging objects and/or ii) obtained by simulating an imaging of objects by the imaging apparatus based on the model of the imaging apparatus, c) reconstructing, based on the model of the imaging apparatus, several training image data sets from the training signal sets, each training image data set comprising image data relating to an optoacoustic and/or ultrasonic image of an object, and d) training the artificial neural network, which comprises an input layer and an output layer, the training comprising i) inputting the training signal sets at the input layer, ii) obtaining, for each inputted training signal set, an output image data set which is outputted at the output layer, and iii) comparing each output image data set with the training image data set which was reconstructed from the respectively inputted training signal set.

Aspects of present disclosure relate to a method and corresponding system for optoacoustic and ultrasonic imaging, a method for reconstructing optoacoustic and ultrasonic images and a method for training an artificial neural network provided therefor.

Optoacoustic imaging, also referred to as “photoacoustic” imaging, requires reconstruction of an initial pressure distribution (p₀) that is induced by laser illumination of biological tissue by suitable image reconstruction algorithms. Clinical in-vivo applications of optoacoustic imaging require high-quality images that resolve details in the tissue contrast, a fast reconstruction of the initial pressure distribution and image display to enable live feedback to the user for dynamic imaging (that is, operations where the user needs real-time image display to position the probe), live tuning of the speed of sound parameter to enable dynamic focusing of the image for different imaged tissue types, and compatibility of the live image reconstruction with high data rates and high image resolution to enable optimal data usage without loss of image quality.

Hybrid optoacoustic and ultrasound (OPUS) systems are configured to acquire both optoacoustic (OA) and ultrasound (US) signals. The associated image reconstruction requires a reconstruction of both the initial optoacoustic pressure distribution (p₀) in optoacoustic imaging and the reflection coefficient distribution (I) in ultrasound imaging. The relation between both these quantities and the recorded OA and US data depends on acoustic properties of the tissue, particularly on the speed of sound distribution (SoS). Optimal usage of OPUS data requires a reconstruction of the acoustic properties (SoS, mechanical density) simultaneously with the two unknowns (p₀, ┌). Commercial usage of the imaging data requires realization of the mapping (US data, OA data) [symbol] (SoS, ┌, p₀) in real time (preferably at least 25 frames per second) in the system hardware to enable live feedback to the system user on the system monitor.

It is an object of present disclosure to provide a method and corresponding system for optoacoustic and ultrasonic imaging, a method for reconstructing optoacoustic and ultrasonic images and a method for training an artificial neural network provided therefor which are improved in view of at least a part of the above-mentioned needs.

This object is achieved by a method for training an artificial neural network for reconstructing optoacoustic and ultrasonic images according to claim 1, a method for reconstructing optoacoustic and ultrasonic images according to claim 10, a method for optoacoustic and/or ultrasonic imaging according to claim 11 and a system for optoacoustic and/or ultrasonic imaging according to claim 12.

A first aspect of present disclosure relates to a, preferably computer-implemented, method for training an artificial neural network for reconstructing optoacoustic and/or ultrasonic images from signals generated by an imaging apparatus for optoacoustic and/or ultrasonic imaging, wherein the method comprises: a) providing a model, which is also referred to as “forward model”, of the imaging apparatus, the model characterizing a relation, which is also referred to as “mapping”, between i) a spatial distribution, also referred to as “image”, of acoustic sources emitting and/or reflecting acoustic waves and ii) signals, also referred to as “sinogram”, “sinograms” or “data”, generated by detection elements of the imaging apparatus upon detecting the acoustic waves, b) providing several training signal sets, also referred to as “training sinograms”, each training signal set comprising a plurality of training signals which were i) generated by the imaging apparatus upon imaging objects and/or ii) obtained by simulating an imaging of objects by the imaging apparatus based on the model of the imaging apparatus, c) reconstructing, based on the model of the imaging apparatus, several training image data sets from the training signal sets, each training image data set comprising image data relating to an optoacoustic and/or ultrasonic image of an object, and d) training the artificial neural network, which comprises an input layer and an output layer, the training comprising i) inputting the training signal sets at the input layer, ii) obtaining, for each inputted training signal set, an output image data set which is outputted at the output layer, and iii) comparing each output image data set with the training image data set which was reconstructed from the respectively inputted training signal set.

Preferably, reconstructing training image data sets from the training signal sets according to step c) of the first aspect includes an implementation of a, preferably iterative, model-based image reconstruction methodology, i.e., a reconstruction methodology which is based on the model (forward model) of the imaging apparatus.

Preferably, reconstructing training image data sets from the training signal sets according to step c) of the first aspect includes an implementation of a model-based solution (i.e. a solution based on the forward model) to the inverse problem, preferably a realization of a mapping (US data, OA data) [symbol] (SoS, ┌, p₀), wherein “US data” corresponds to signals (sinogram(s)) generated by the detection elements upon detecting acoustic waves reflected by the acoustic sources, “OA data” corresponds to signals (sinogram(s)) generated by the detection elements upon detecting acoustic waves emitted by the acoustic sources, “SoS” corresponds to a speed of sound distribution, reflection coefficient distribution ┌ corresponds to an ultrasonic (US) image, and initial pressure distribution p₀ corresponds to an optoacoustic (OA) image.

Preferably, in step c) of the first aspect the training image data sets can be reconstructed from training signal sets which are or were obtained by imaging objects and/or by simulating an imaging of objects according to step b). In other words, the training image data sets can be reconstructed from training signal sets which are or were i) generated by the imaging apparatus upon imaging real objects, or ii) obtained by simulating an imaging of objects. Alternatively, a part of the training signal sets can be generated according to i) and another part of the training signal sets can be obtained according to ii).

Preferably, in step b ii) of the first aspect at least some of the training signal sets comprise training signals which were obtained (synthesized) by simulating an imaging of objects by the imaging apparatus for optoacoustic and/or ultrasonic imaging based on i) the model (forward model) of the imaging apparatus for optoacoustic and/or ultrasonic imaging and ii) initial images of objects which were obtained by any imaging apparatus (which is preferably different from the imaging apparatus for optoacoustic and/or ultrasonic imaging). In other words, in step b ii) the model of the imaging apparatus for optoacoustic and/or ultrasonic imaging as provided in step a) transforms the initial images into the training signal sets, wherein each of the initial images is considered as a spatial distribution of acoustic sources emitting and/or reflecting acoustic waves which is transformed into a training signal set (training sinogram) which is considered as being generated by the detection elements of the imaging apparatus for optoacoustic and/or ultrasonic imaging upon detecting the acoustic waves. For example, the initial images can be photographs, e.g. from arbitrary objects and/or scenes, taken by a conventional camera, or medical and/or in-vivo images obtained by a medical and/or tomographic imaging modality, for example a CT, MRT, ultrasonic and/or optoacoustic imaging modality. A second aspect of present disclosure relates to a, preferably computer-implemented, method for reconstructing an optoacoustic and/or ultrasonic image from a set of signals, which is also referred to as “sinogram” or may be or comprise a “set of sinograms”, generated by an imaging apparatus for optoacoustic and/or ultrasonic imaging, the method comprising: inputting the set of signals at an input layer of the artificial neural network which has been trained by the method according to the first aspect, and obtaining at least one optoacoustic and/or ultrasonic image which is outputted at an output layer of the trained artificial neural network.

A third aspect of present disclosure relates to a, preferably computer-implemented, method for optoacoustic and/or ultrasonic imaging comprising: irradiating an object with electromagnetic and/or acoustic waves and generating a set of signals, which is also referred to as “sinogram” or may be or comprise a “set of sinograms”, by detecting acoustic waves emitted and/or reflected by the object in response thereto by means of an imaging apparatus for optoacoustic and/or ultrasonic imaging, and reconstructing an optoacoustic and/or ultrasonic image of the object from the set of signals by the method according to the second aspect.

A fourth aspect of present disclosure relates to a system for optoacoustic and/or ultrasonic imaging comprising an imaging apparatus for optoacoustic and/or ultrasonic imaging, the imaging apparatus comprising an irradiation device configured to irradiate an object with electromagnetic and/or acoustic waves and a detection device configured to generate a set of signals, which is also referred to as “sinogram” or may be or comprise a “set of sinograms”, by detecting acoustic waves emitted and/or reflected by the object in response to irradiating the object with the electromagnetic and/or acoustic waves, and a processor configured to reconstruct an optoacoustic and/or ultrasonic image of the object from the set of signals by inputting the set of signals at an input layer of an artificial neural network which has been trained by the method according to the first aspect, and obtaining at least one optoacoustic and/or ultrasonic image which is outputted at an output layer of the trained artificial neural network.

Preferably, in particular in relation to the second to fourth aspect, in the case that the set of signals comprises a single sinogram, a single-wavelength optoacoustic image can be reconstructed therefrom. It is preferred that the set of signals comprises a set of sinograms (i.e. the set of sinograms comprises several sinograms) from which, e.g., multi-wavelength optoacoustic image(s) and/or ultrasonic image(s), including superimposed and/or co-registered and/or “hybrid” optoacoustic image(s) and/or ultrasonic image(s), can be reconstructed or obtained, respectively.

A fifth aspect of present disclosure relates to a computer program product causing a computer, computer system and/or distributed computing environment to execute the method according to i) the first aspect of present disclosure and/or ii) the second aspect of present disclosure and/or iii) the third aspect of present disclosure.

A sixth aspect of present disclosure relates to a computer, computer system and/or distributed computing environment (e.g. a client-server system or storage and processing resources of a computer cloud) comprising means for carrying out the method according to i) the first aspect of present disclosure and/or ii) the second aspect of present disclosure and/or iii) the third aspect of present disclosure.

A seventh aspect of present disclosure relates to a computer-readable storage medium having stored thereon instructions which, when executed by a computer, computer system or distributed computing system, cause same to carry out the method according to i) the first aspect of present disclosure and/or ii) the second aspect of present disclosure and/or iii) the third aspect of present disclosure.

Another aspect of present disclosure relates to a computer program product comprising instructions causing the processor of the system according to the fourth aspect to execute the steps of the method according to the first aspect.

Yet another aspect of present disclosure relates to a computer program product comprising instructions causing the system according to the fourth aspect to execute the steps of the method according to the second aspect and/or the third aspect.

Preferably, within the meaning of present disclosure, the term “acoustic source” relates to any entity contained in an imaged object which emits ultrasound (in response to stimulating the entity with pulsed electromagnetic radiation) and/or which reflects ultrasound (in response to applying ultrasound to the entity).

Preferred aspects of present disclosure are based on the approach of providing a dedicated method for training an artificial neural network, also referred to as “deep learning” and “deep neural network”, respectively, and using the trained artificial neural network for reconstructing optoacoustic and/or ultrasonic images from signals generated by an imaging apparatus for optoacoustic and/or ultrasonic imaging. A first implementation, herein also referred to as “DeepMB”, relates to a deep learning solution for optoacoustic (OA) image reconstruction, preferably with tunable speed of sound (SoS). A second implementation, herein also referred to as “DeepOPUS”, relates to a deep learning solution for simultaneous or joint (and, therefore, synergistic) reconstruction of co-registered optoacoustic (OA) and ultrasonic (US) images, preferably including speed of sound distribution (SoS) retrieval.

Preferably, in the DeepMB implementation a comprehensive dataset of input-output pairs (OA data, SoS) [symbol] (OA image) is provided and/or generated based on i) a precise modelling and simulation of the OA response (also referred to as OA data) of the imaging system (forward model), and ii) an implementation of an iterative model-based image reconstruction methodology. Then, a deep neural network is trained with the afore-mentioned data set. That is, the target reference used during the training is the model-based reconstruction of the corresponding optoacoustic image. The obtained trained neural network can then be implemented in a system hardware, e.g. via dedicated graphical processing units, and/or used for optoacoustic image reconstruction, wherein a set of optoacoustic signals (sinogram) acquired by an optoacoustic imaging apparatus is inputted at an input layer of the trained neural network, and an optoacoustic image is obtained at an output layer of the trained neural network. In this way, it is possible to reconstruct high-quality OA images with arbitrary content at a high framerate of at least 24 fps and to dynamically (“on-the-fly”) change the SoS during imaging.

Similarly, the DeepOPUS implementation preferably includes i) a precise modelling and simulation of the OA and US responses (also referred to as “OA data” and “US data”, respectively) of the imaging system (forward model), and ii) an implementation of a model-based solution (based on the afore-mentioned forward model) to the inverse problem, i.e., realization of the mapping (US data, OA data) [symbol] (SoS, ┌, p₀), wherein the reflection coefficient distribution I corresponds to an ultrasonic (US) image, and the initial pressure distribution p₀ corresponds to an optoacoustic (OA) image. A comprehensive data set of input-output pairs ((US data, OA data), (SoS, ┌, p₀)) is provided and/or generated, preferably via simulation of US data and OA data, e.g. from a public general feature image database, and reconstruction of (SoS, ┌, p₀) with the method (model-based solution) set forth in the afore-mentioned item ii). A deep neural network is trained with the afore-mentioned data set. That is, the target references used during the training are the model-based reconstructions of the corresponding optoacoustic and ultrasound images. The obtained trained neural network can then be implemented in a system hardware, e.g. via dedicated graphical processing units, and/or used for optoacoustic and ultrasonic image reconstruction, wherein a set of optoacoustic and ultrasonic signals (sinograms) acquired by an optoacoustic and ultrasonic imaging apparatus is inputted at an input layer of the trained neural network, and an optoacoustic and ultrasonic image is obtained at an output layer of the trained neural network. In this way, it is possible to properly utilize the combination of OA and US data simultaneously or jointly (as opposed to “one after the other”) to i) quantify the pixel-wise SoS distribution in the imaged region, ii) correct for reflection artifacts in both OA and US images, obtain a high framerate of at least 24 fps, and iii) improve the image quality via the synergistic effects of OA and US data integration.

In summary, by means of present disclosure the quality of the simultaneously or jointly reconstructed OA and US images is improved, and high frame rates are achieved. In particular, present disclosure allows for correcting image artifacts in both optoacoustic and ultrasonic images and quantifying and/or dynamically changing the speed of sound distribution in the imaged region.

Preferably, the artificial neural network is a convolutional neural network (CNN), in particular U-Net.

Preferably, the model characterizes at least one of the following: i) a propagation of the acoustic waves from the acoustic sources towards the detection elements, ii) a response of the detection elements upon detecting the acoustic waves, and/or iii) a noise of the imaging apparatus.

Preferably, characterizing the propagation of the acoustic waves includes at least one of the following: i) an acoustic wave propagation model, which is the same for the propagation of both emitted optoacoustic waves and reflected ultrasound waves, ii) a propagation of the acoustic waves through a medium with an inhomogeneous speed of sound distribution, and/or iii) a reflection of the acoustic waves at one or more reflective interfaces in the medium investigated.

Preferably, at least some of the training signal sets comprise training signals which were obtained (synthesized) by simulating an imaging of objects by the imaging apparatus for optoacoustic and/or ultrasonic imaging based on i) the model of the imaging apparatus for optoacoustic and/or ultrasonic imaging and ii) initial images of objects which were obtained by any imaging apparatus, which is preferably different from the imaging apparatus for optoacoustic and/or ultrasonic imaging. For example, the initial images can be photographs, e.g. “real-world images”, from arbitrary objects and/or scenes taken by a conventional camera. Alternatively or additionally, the initial images can be medical and/or in-vivo images obtained by a medical and/or tomographic imaging modality, for example a CT, MRT, ultrasound and/or optoacoustic imaging modality.

In this way, the model (forward model) of the imaging apparatus for optoacoustic and/or ultrasonic imaging transforms the initial images into the training signal sets, wherein each of the initial images is considered as a spatial distribution of acoustic sources contained in the object represented in the initial image and emitting and/or reflecting acoustic waves. In other words, the spatial distribution of acoustic sources is transformed into a training signal set (training sinogram) which can, therefore, be considered as having been (notionally) generated by the detection elements of the imaging apparatus upon (notionally) detecting the acoustic waves (notionally) emitted and/or reflected by the acoustic sources considered to be contained in the object represented in the initial image.

In this way, a plurality of training signal sets can be easily and quickly generated based on any available initial image, rather than generating the training signal sets by imaging objects with the imaging apparatus itself.

Preferably, in particular in the DeepMB implementation, reconstructing a training image data set x* from a training signal set s comprises: i) calculating, based on the model M of the imaging apparatus, several prediction signal sets M(x) from several varying image data sets x, ii) calculating, for each of the varying image data sets x, a first distance metric d(M(x), s) between the respective prediction signal set M(x) and the training signal set s, and iii) determining the image data set x* for which the first distance metric d(M(x), s) between the respective prediction signal set M(x) and the training signal set s exhibits a minimum, wherein the reconstructed training image data set is the determined image data set x*. Preferably, with M(x) as the model that maps an image x to a sinogram s and d as a distance metric for sinograms, the reconstruction of an image x″ is given by:

$${x}^{*} = arg {min}_{x} d ( M ( x ) , s ) .$$

Thus, the varying image data sets x can be considered as different values of the argument x of the function M(x) when iteratively (i.e. by varying the values of the argument x) determining the value x* of the argument x for which the distance metric d(M(x), s) has a minimum.

Preferably, in particular in the DeepOPUS implementation, each training signal set s_{OA}, s_{US} comprises a plurality of optoacoustic training signals s_{OA} and a plurality of ultrasonic training signals s_{US}. At least one training image data set x*_{OA}, X*_{US}, and optionally c*, is reconstructed from at least one training signal set s_{OA}, s_{US} based on a simultaneous and/or joint consideration of the respective optoacoustic training signals s_{OA} and ultrasonic training signals s_{US} comprised in the at least one training signal set s_{OA}, s_{US}. In this way, the artificial neural network is trained for a simultaneous and/or joint optoacoustic and ultrasonic image reconstruction in which OA and US data are advantageously combined (as opposed to be processed “one after the other”), so as to allow for, e.g. a quantification of a pixel-wise SoS distribution in the imaged region, a correction for reflection artifacts in both OA and US images, obtaining a high framerate of at least 24 fps, and improving the image quality via synergistic effects of OA and US data integration. In other words, US image reconstruction is improved by considering information from OA data and/or OA image(s), and OA image reconstruction is improved by considering information from US data and/or US image(s).

Preferably, in particular in the DeepOPUS implementation, reconstructing at least one training image data set X*_{OA}, x*_{US}, c* from at least one training signal set s_{OA}, s_{US}) comprises: i) calculating, based on the model M_{c} of the imaging apparatus taking into account a propagation of the acoustic waves through a medium with a, in particular pre-defined or reconstructed, speed of sound distribution c, several prediction signal sets M_{c}(x_{OA}, x_{US}) from several varying image data sets x_{OA}, x_{US}, ii) calculating, for each of the varying image data sets x_{OA}, x_{US}, a second distance metric d(M_{c}(x_{OA}, x_{US}), (s_{OA}, s_{US})) between the respective prediction signal set M_{c}(x_{OA}, x_{US}) and the training signal set s_{OA}, s_{US}, and iii) determining at least one image data set X*_{OA}, x*_{US}, c* for which the second distance metric d(M_{c}(x_{OA}, x_{US}), (s_{OA}, s_{US}) between the respective prediction signal set d(M_{c}(x_{OA}, x_{US}) and the at least one training signal set s_{OA}, s_{US} exhibits a minimum, wherein the at least one training image data set is the at least one determined image data set x_{OA}, x*_{US}, c*. Preferably, the speed of sound distribution c can be an inhomogeneous or homogeneous speed of sound distribution. Preferably, with M_{c}(x_{OA}, x_{US}) as the model that maps optoacoustic and ultrasound images x_{OA}, x_{US} to optoacoustic and ultrasound sinograms s_{OA}, s_{US} and d as a distance metric for the sinograms, the reconstruction of images x_{OA}, x*_{US}, and optionally c*, is given by:

$$( {x}_{OA}^{*} , {x}_{US}^{*} , {c}^{*} ) = arg {min}_{( {x}_{OA} , {x}_{Us} , c )}$$

$$d ( {M}_{c} ( {x}_{OA} , {x}_{U S} ) , ( {s}_{OA} , {s}_{U S} ) ) .$$

Thus, the varying image data sets x_{OA}, x_{US}, and optionally c, can be considered as different values of the arguments x_{OA}, x_{US} of the function M_{c}(x_{OA}, x_{US}) when iteratively (i.e. by varying the values of the arguments x_{OA}, x_{US}) simultaneously or jointly determining the values X*_{OA}, x*_{US}, and optionally c*, of the arguments x_{OA}, x_{US}, and optionally c, for which the distance metric d(M_{c}(x_{OA}, x_{US}), (s_{OA}, s_{US}) has a minimum.

Preferably, comparing the output image data set with the respective training image data set X*_{OA}, x*_{US}, c* comprises determining a loss function which is given by: a third distance metric, in particular a means squared error, between the output image data set, on the one hand, and the respective training image data set x*_{OA}, x*_{US} and speed of sound distribution c* reconstructed from the respective training signal set, on the other hand, and/or the first and/or second distance metric which is applied to the output image data set.

Preferably, the at least one artificial neural network is given by i) a single deep neural network or ii) a cascade of multiple (N) deep neural networks.

Preferably, in a so-called one-step process, the training comprises i) inputting the training signal sets s_{OA}, s_{US} at the input layer, ii) obtaining, for each inputted training signal set s_{OA}, s_{US}, both the output image data set x_{OA}, x_{US} and an output speed of sound distribution c which are outputted at the output layer, and iii) comparing each output image data set x_{OA}, x_{US} and output speed of sound distribution c with the training image data set X*_{OA}, x*_{US} and, respectively, a training speed of sound distribution c* which were reconstructed from the respectively inputted training signal set s_{OA}, s_{US}.

Preferably, in a so-called two-step process, the training comprises i) inputting the training signal sets s_{OA}, s_{US} at the input layer, ii) obtaining, for each inputted training signal set s_{OA}, s_{US}, an output speed of sound distribution c which is outputted at the output layer, and iii) comparing each output speed of sound distribution c with a training speed of sound distribution c* which was reconstructed from the respectively inputted training signal set s_{OA}, s_{US}, and subsequently i) inputting the training signal sets s_{OA}, s_{US} and the output speed of sound distribution c at the input layer, ii) obtaining, for each inputted training signal set s_{OA}, s_{US} and output speed of sound distribution c, the output image data set x_{OA}, x_{US} which is outputted at the output layer, and iii) comparing each output image data set x_{OA}, x_{US} with the training image data set X*_{OA}, x*_{US} which was reconstructed from the respectively inputted training signal set s_{OA}, s_{US}.

Preferably, each training signal set may comprise several training sinograms and/or a set of training sinograms (i.e. the set of training sinograms comprises several training sinograms) so as to particularly train the artificial neural network for reconstructing and/or obtaining, e.g., multi-wavelength optoacoustic image(s) and/or ultrasonic image(s), including superimposed and/or co-registered and/or “hybrid” optoacoustic image(s) and/or ultrasonic image(s).

Preferably, in particular in the DeepOPUS implementation of a method for reconstructing an optoacoustic and ultrasonic image x_{OA}, x_{US} from a set of signals s_{OA}, s_{US} generated by the imaging apparatus, the optoacoustic signals s_{OA} and ultrasonic signals s_{US} comprised by the set of signals s_{OA}, s_{US} are simultaneously and/or jointly inputted at the input layer of the trained artificial neural network, and/or the optoacoustic image x_{OA} and ultrasonic image x_{US} are simultaneously and/or jointly outputted at the output layer of the trained artificial neural network.

In this way, a simultaneous and/or joint optoacoustic and ultrasonic image reconstruction is performed (as opposed to reconstructing the optoacoustic and ultrasonic image “one after the other”), in which OA and US data are advantageously combined, e.g. to quantify the pixel-wise SoS distribution in the imaged region, correct for reflection artifacts in both OA and US images, obtain a high framerate of at least 24 fps, and improve the image quality via synergistic effects of OA and US data integration. In other words, US image reconstruction is improved by considering information from OA data and/or OA image(s), and OA image reconstruction is improved by considering information from US data and/or US image(s).

Other preferred and/or alternative aspects and/or embodiments of present disclosure are discussed in the following, in particular with reference to the DeepOPUS and DeepMB implementation.

### DeepOPUS

Preferably, the DeepOPUS implementation includes correctly modeling, learning and inferring a mapping between ultrasound and optoacoustic signals (sinogram(s)), on the one hand, and an ultrasound and optoacoustic image and a speed of sound (SoS) distribution, on the other hand: {ultrasound signals, optoacoustic signals}→{ultrasound image, optoacoustic image, speed of sound distribution}.

Preferably, the DeepOPUS implementation includes at least one of the following aspects or a combination thereof: 1) providing a forward model that simulates the physics, in particular the acoustic propagation path, and data acquisition of the optoacoustic and ultrasound imaging process, 2) providing an inverse problem solver that reconstructs optoacoustic and ultrasound images as well as the speed of sound distribution from the optoacoustic and ultrasound signal data using the forward model, and 3) providing a deep learning solution that implements the inverse problem solver in real time on the system, where the forward model can be used to generate, preferably synthetic, training data obtained from a, for example real-world, image dataset.

In the following, preferred embodiments of the above-mentioned aspects 1) to 3) are described.

### 1) The Forward Model

Preferably, the forward model
a. simulates acoustic wave propagation for both optoacoustic and ultrasound waves with the same acoustic physics model (i.e. the optoacoustic and ultrasound model are compatible, and/or
b. simulates acoustic wave propagation through media with inhomogeneous speed of sound distributions, and/or
c. simulates (at least one) reflection event(s) at reflective interfaces in the medium.

Feature a. is preferred to couple the information contained in the two different OA and US imaging modalities. Features b. and c. are preferred to allow speed of sound inference.

Preferably, the forward model of the acoustic components of the system takes into account different aspects: (i) the physics of acoustic wave propagation, (ii) the conversion of acoustic pressure to electrical signals by the detectors, (iii) the system noise. In summary, it provides a function that maps images (or volumes) of initial pressure/reflectivity data to simulated optoacoustic/ultrasound sinograms.

Regarding (i), the model needs to approximate solutions to the acoustic wave equation

$$Δ p - \frac{1}{{c}^{2}} \frac{{\partial }^{2}}{\partial {t}^{2}} p = 0 ,$$

with p the pressure, t the time, and c the speed of sound distribution.

For the optoacoustic part, an initial value problem needs to be solved with p(x,0)=p₀ the initial pressure distribution. The second initial condition d/dt p(x,0)=0 is a result of the fast optical absorption process in optoacoustics.

For the ultrasound part, the inhomogeneous wave equation needs to be solved with a source term that describes the acoustic excitation of tissue. There are several algorithmic options for solving these problems in a common framework: finite element solvers, pseudo-spectral methods (e.g., k-wave), geometric acoustics solvers (e.g., raytracing).

Regarding (ii), the pressure recorded at the detectors is preferably described by the total impulse response of the system, i.e., the signal generated by an impulse (infinitesimally small acoustic source) located in the field of view of the system. If the system is linear, the impulse response fully characterizes the output of the system. The experimental and computational characterization of the impulse response and its integration into a forward model is described in detail in the following publications, which are incorporated by reference herewith: Chowdhury, K. B., Prakash, J., Karlas, A., Jüstel, D. & Ntziachristos, V. A Synthetic Total Impulse Response Characterization Method for Correction of Hand-Held Optoacoustic Images. IEEE Trans Med Imaging 39, 3218-3230 (2020), and Chowdhury, K. B., Bader, M., Dehner, C., Justel, D. & Ntziachristos, V. Individual transducer impulse response characterization method to improve image quality of array-based handheld optoacoustic tomography. Opt Lett 46, 1-4 (2021).

Regarding (iii), noise can preferably be incorporated as random distortions of the data, if a probabilistic model of the noise is available. It can also be integrated from measured pure system noise, for example, when the noise is signal independent and additive. Often noise is not explicitly included into the model and dealt with during the image reconstructions (e.g., with suitable regularization schemes for variational formulations of the inverse problem; see below). Details on electrical noise in optoacoustic imaging can be found in the following publication, which is incorporated by reference herewith: Dehner, C., Olefir, I., Basak Chowdhury, K., Justel, D. and Ntziachristos, V. Deep learning based electrical noise removal enables high spectral optoacoustic contrast in deep tissue. arXiv: 2102.12960 (2021).

### 2) The Inverse Problem Solver

Preferably, the inverse problem solver
a. approaches the inverse problem via a variational formulation of the problem (also called model-based approach), i.e., a distance metric between the data and the prediction of the forward model is minimized to recover the unknowns (namely, ultrasound image, optoacoustic image, speed of sound distribution), and/or
b. addresses the nonlinearity and potential ill-posedness of the problem, preferably with integration of prior information and other regularization techniques, and with suitable strategies for initial guesses.

Feature a. represents the preferred use of the most powerful framework for inverse problems. Feature b. provides optimal performance of the solver.

Preferably, the inverse problem solver is an implementation of an algorithm that approximates the image/volume data, given the sinogram data acquired in optoacoustic or ultrasound imaging.

Preferably, a very general and powerful approach to inverse problems is the variational formulation of the inverse problem. The idea is to minimize a distance metric between the acquired signals and the prediction of the forward model (see above) over a set of possible solutions. The inverse problem can then be tackled with solvers for optimization problems (e.g., gradient descent methods). If M is the model that maps an image x to a sinogram s, and d is a distance metric for sinograms, then an image reconstruction x* is, for example, given by

$${x}^{*} = arg {min}_{x} d ( M ( x ) , s ) .$$

In the case of DeepOPUS, the unknowns—speed of sound distribution c, optoacoustic image x_{OA}, and ultrasound image x_{US}—are reconstructed in the same way. The dependence of the model on the speed of sound is indicated by writing M_{c}. Note that the model M_{c} has two outputs for DeepOPUS: an optoacoustic sinogram s_{OA} and ultrasound sinogram s_{US}. The metric d, thus, needs to quantify the distance between the collections of sinograms:

$$( {x}_{OA}^{*} , {x}_{US}^{*} , {c}^{*} ) = arg {min}_{( {x}_{OA} , {x}_{Us} , c )}$$

$$d ( {M}_{c} ( {x}_{OA} , {x}_{U S} ) , ( {s}_{OA} , {s}_{U S} ) ) .$$

Such non-linear minimization problems can be solved with specific solvers for certain choices of d (e.g., non-linear least squares) or more generally with gradient descent methods or Newton's method. If the objective function is convex in the unknowns, then every local minimum is global, such that the latter methods can be used to find a solution. In non-convex situations, a suitable initial guess is necessary to converge to a global minimum.

An additional complication may be ill-posedness, which in present case means the non-uniqueness of the solution. This issue can be addressed by using appropriate assumptions, and incorporate an additional regularization term that enforces well-posedness and therefore unicity of the solution.

### 3) The Deep Learning Solution

Preferably, for the deep learning solution at least one of the following applies:
a. During training, the artificial neural network learns the mapping and has as inputs the optoacoustic and ultrasound signal data, and as output the optoacoustic and ultrasound images, and the speed of sound distribution.
b. The input of the artificial neural network for the training is a synthetic dataset of simulated optoacoustic and ultrasound data, obtained by using the forward model onto a dataset of real-world images. The target reference for the training are the corresponding ground-truth reconstructions of optoacoustic and ultrasound images, together with the speed of sound distribution, obtained by applying the inverse problem solver onto the input training data.
c. When deployed to acquired optoacoustic and ultrasound signal data, the trained artificial neural network applies the learned mapping that has as inputs the acquired optoacoustic and ultrasound signal data, and as output the optoacoustic and ultrasound images, and the speed of sound distribution in real time.

Features a. and b. ensure that a well-defined operator is learned, such that the model generalizes properly to unseen data with minimal dependence on the specifics of the training data set. Feature c. provides the performance which is preferred for real time feedback to the system operator.

The deep learning solution speeds up the solution from the inverse problem solver for on-device application during imaging. It infers the reconstructed OA and US images and the SoS distribution from the recorded OA and US sinograms of a scan.

Preferably, the training data are OA and US sinograms that were synthesized with the forward model (see 1) based on suitable real-world images, and corresponding ground truth reference data (OA and US images, SoS distribution) obtained from the inverse problem solver (see 2).

Preferably, the deep learning solution employs one of the following options as loss function during training:
I. A suitable distance metric (e.g., mean squared error) between the output of the employed deep neural network and the ground truth images and SoS distribution obtained from the inverse problem solver (see 2).
II. All or parts of the distance metrics that are used in the variational formulation of the inverse problem solver in 2, applied to the output of the employed deep neural network.
III. A combination of I and II.

### Deep Neural Network Architecture

Preferably, the deep learning solution infers the OA and US images and the SoS distribution either in a one-step or in a two-step process using a single or a cascade of N deep neural networks, respectively. Further details can be found, for example, in the following publication which is incorporated by reference herewith: Hauptmann, A., Lucka, F., Betcke, M., Huynh, N., Adler, J., Cox, B., Beard, P., Ourselin, S. and Arridge, S. Model-Based Learning for Accelerated, Limited-View 3-D Photoacoustic Tomography. IEEE Trans Med Imaging 37, 1382-1393 (2018),

One-step process with single deep neural network:
(OA_sinogram, US_sinogram)→_{DNN1} (OA_image, US_image, SoS_distribution)

One-step process with cascade of N deep neural networks:

(OA_sinogram, US_sinogram, OA_image^{(init)}, US_image^{(init)}, SoS_distribution^{(init)})
→_{DNN1}⁽¹⁾ (OA_image⁽¹⁾, US_image⁽¹⁾, SoS_distribution⁽¹⁾)
(OA_sinogram, US_sinogram, OA_image⁽¹⁾, US_image⁽¹⁾, SoS_distribution⁽¹⁾)
→_{DNN1}⁽²⁾ (OA_image⁽²⁾, US_image⁽²⁾, SoS_distribution⁽²⁾)
...
(OA_sinogram, US_sinogram, OA_image^{(N-1)}, US_image^{(N-1)}, SoS_distribution^{(N-1)})
→_{DNN1}^{(N)} (OA_image^{(N)}, US_image^{(N)}, SoS_distribution^{(N)})

Two-step process with single deep neural network:
(OA_sinogram, US_sinogram)→_{DNN1} (SoS_distribution)
followed by:
(OA_sinogram, US_sinogram, SoS_distribution)→_{DNN2} (OA_image, US_image)

Two-step process with cascade of N deep neural networks:

(OA_sinogram, US_sinogram, SoS_distribution^{(init)}) →_{DNN1}⁽¹⁾ (SoS distribution⁽¹⁾)
(OA_sinogram, US_sinogram, SoS distribution⁽¹⁾) →_{DNN1}⁽²⁾ (SoS distribution⁽²⁾)
...
(OA_sinogram, US_sinogram, SoS_distribution^{(N-1)}) →_{DNN1}^{(N)} (SoS distribution^{(N)})

followed by

(OA_sinogram, US_sinogram, SoS_distribution, OA_image^{(init)}, US_image^{(init)})
→_{DNN2}⁽¹⁾ (OA_image⁽¹⁾, US_image⁽¹⁾
(OA_sinogram, US_sinogram, SoS_distribution, OA_image⁽¹⁾, US_image⁽¹⁾)
→_{DNN2}⁽²⁾ (OA_image⁽²⁾, US_image⁽²⁾)
...
(OA_sinogram, US_sinogram, SoS_distribution, OA_image^{(N-1)}, US_image^{(N-1)})
→_{DNN2}^{(N)} (OA_image^{(N)}, US_image^{(N)}
OA_image^{(init)}, US_image^{(init)}, SoS_distribution ^{(init)} are initial guesses that are pref-
erably derived at random, from the OA and US sinograms, or from further prior
information.

If not specified otherwise, “OA_image”, “US_image”, and “SoS_distribution” correspond to the final version, namely “OA_image^{(N)}”, “US_image^{(N)}”, and “SoS_distribution (N)”.

Preferably, the employed deep neural networks (denoted as DNNₓ above) are Unet-like convolutional neural networks or ViT-like transformers (Vision transformers), as exemplarily described in the following publication which is incorporated by reference herewith: Dosovitskiy, A., Beyer, L., Kolesnikov, A., Weissenborn, D., Zhai, X., Unterthiner, T., Dehghani, M., Minderer, M., Heigold, G., Gelly, S., Uszkoreit, J. and Houlsby, N. An Image is Worth 16×16 Words: Transformers for Image Recognition at Scale. arXiv: 2010.11929 (2020).

Alternatively, two baseline deep neural networks can be defined, which are simpler and less potent that the DeepOPUS network described above:
a so-called DeepMB network, which can learn and subsequently infer the mapping:

### (OA_sinogram, SoS_distribution)→DeepMB (OA_image)

a so-called DeepUS network, which can learn and subsequently infer the mapping:

### (US_sinogram, SoS_distribution)→Deepus (US_image)

### DeepMB

Preferably, the DeepMB implementation includes correctly modeling, learning and inferring a mapping between optoacoustic signals (sinogram(s)), on the one hand, and an optoacoustic image and a speed of sound (SoS) distribution, on the other hand: {optoacoustic signals}→{optoacoustic image, speed of sound distribution}.

As DeepMB is applied to optoacoustic image reconstruction only, it can be—at least to some extent—considered as a special case of the DeepOPUS implementation, which is applied to both optoacoustic and ultrasonic image reconstruction. Therefore, the above explanations regarding the DeepOPUS implementation apply accordingly to the DeepMB implementation.

Further advantages, features and examples of the present disclosure invention will be apparent from the following description of following figures:

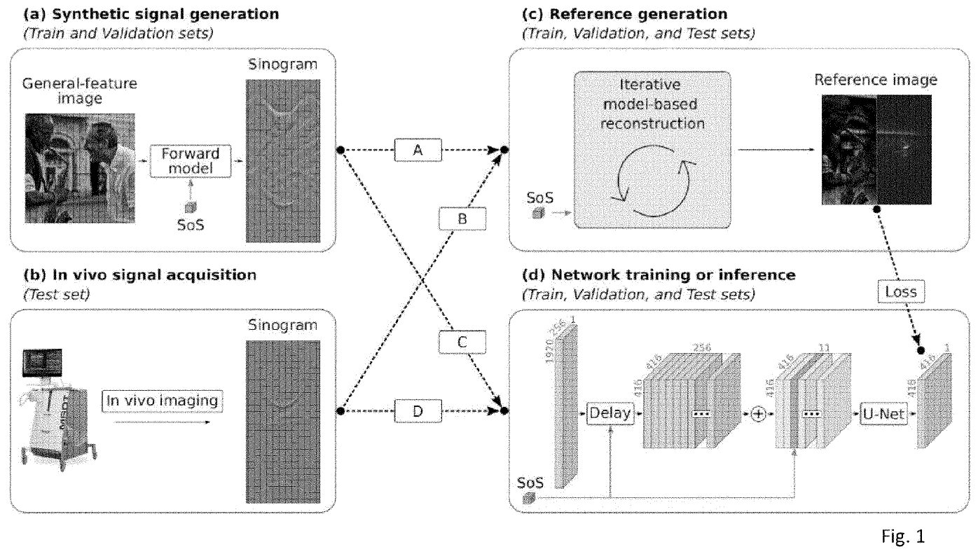

FIG. 1 shows a schematic of an example of a DeepMB pipeline for training, in vivo imaging and evaluation;

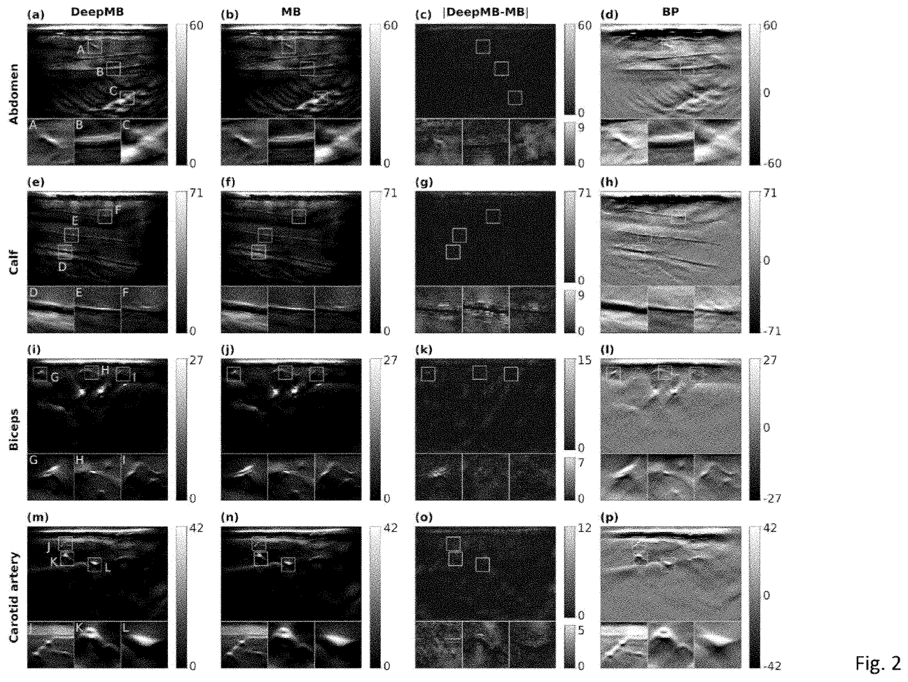

FIG. 2 shows examples of an in vivo test dataset for different anatomical locations;

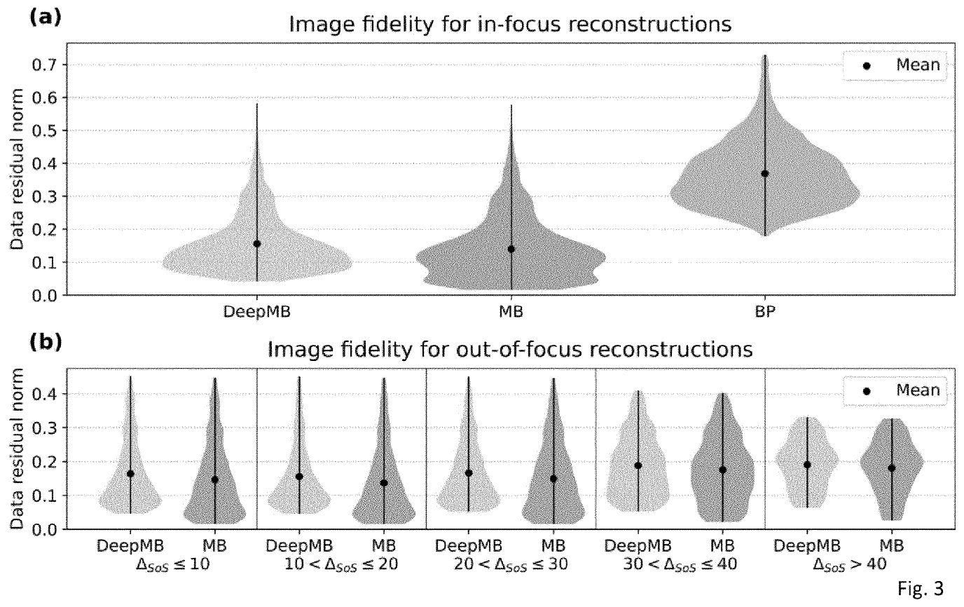

FIG. 3 shows data residual norms of optoacoustic images from deep model-based (DeepMB), model-based (MB), and backprojection (BP) reconstruction;

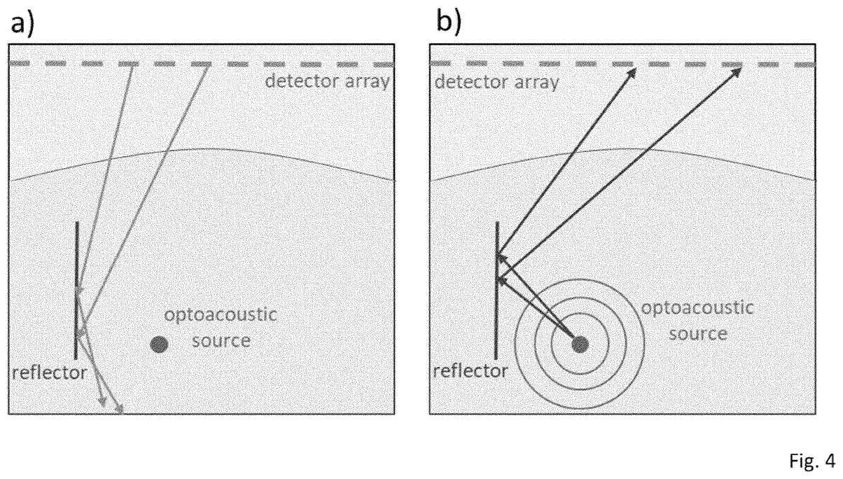

FIG. 4 shows a schematic representation to illustrate an improved US image reconstruction of reflective interfaces with the DeepOPUS implementation;

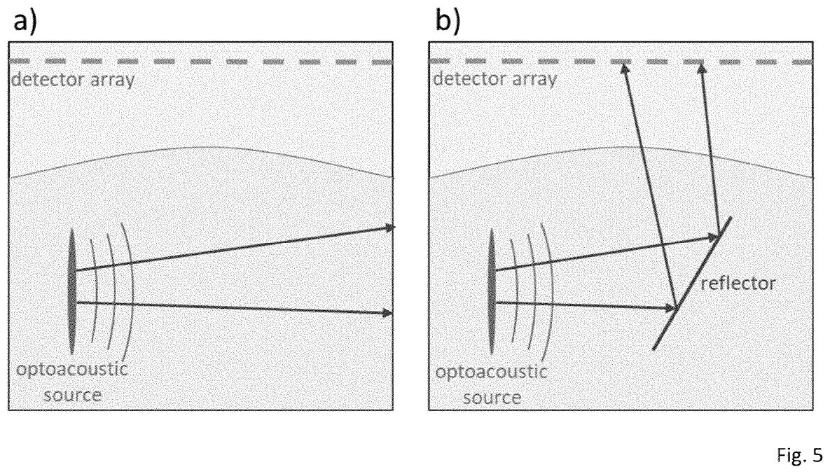

FIG. 5 shows a schematic representation to illustrate an improved OA image reconstruction (initial pressure distribution) with the DeepOPUS implementation;

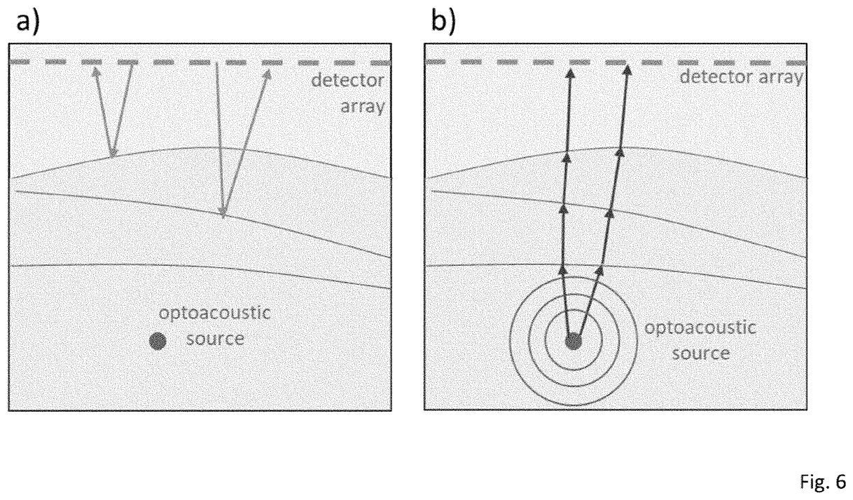

FIG. 6 shows a schematic representation to illustrate an improved reconstruction of the speed of sound distribution with the DeepOPUS implementation;

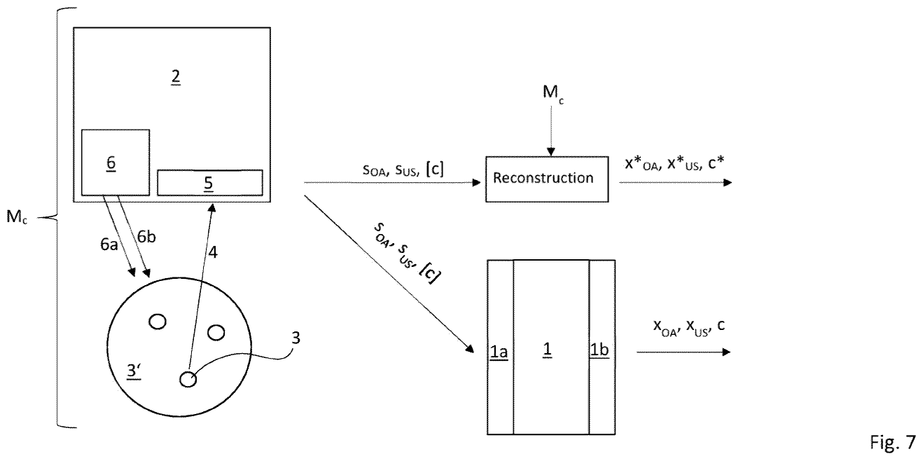

FIG. 7 shows an exemplary schematic to illustrate aspects of present disclosure, in particular a method for training an artificial neural network and a method and corresponding system for optoacoustic and ultrasonic imaging; and

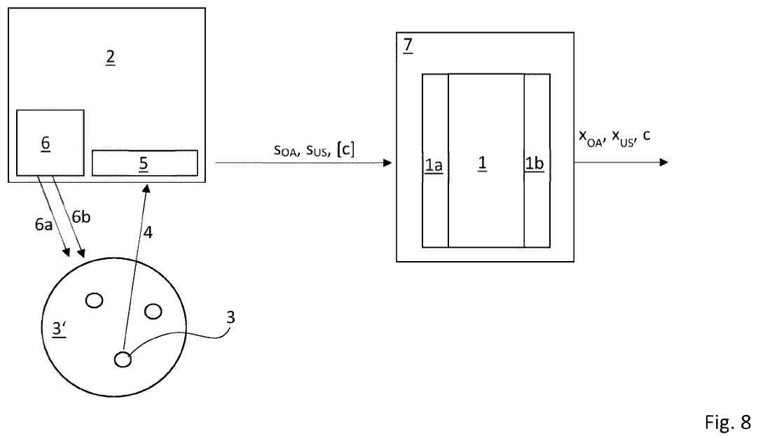

FIG. 8 shows an exemplary schematic to illustrate aspects of present disclosure, in particular a method and corresponding system for optoacoustic and ultrasonic imaging.

### DEEPMB

In the following, examples of a preferred deep-learning model-based optoacoustic reconstruction framework for the DeepMB implementation are shown. Although DeepMB is applied to optoacoustic image reconstruction only, it can be considered as a special case of the DeepOPUS implementation, which is applied to both optoacoustic and ultrasonic image reconstruction. Therefore, the following explanations regarding the DeepMB implementation apply accordingly to the DeepOPUS implementation, which is discussed in more detail further below.

Advantageously, an image quality indistinguishable from state-of-the-art iterative model-based reconstructions at speeds enabling live imaging (100 fps or <10 ms/image, versus 30-60 s/image for iterative model-based reconstruction) can be obtained. Further, DeepMB overcomes the shortcomings of previous deep-learning reconstruction approaches to generalize from preferably synthetic training data to experimental test data by training on optoacoustic signals that are preferably synthesized from a publicly available dataset of real-world images, while using as ground-truth the optoacoustic images generated via model-based reconstruction of the corresponding signals. This training scheme enables DeepMB to learn an accurate and universally applicable model-based optoacoustic reconstruction operator. Further, DeepMB supports dynamic adjustments of the SoS parameter during imaging, which enables the reconstruction of in-focus images for arbitrary tissue types. In contrast to other attempts at applying deep-learning to optoacoustic reconstruction, DeepMB is directly compatible with state-of-the-art clinical MSOT (multi-spectral optoacoustic tomography) scanners because it supports high throughput data acquisition (sampling rate: 40 MHz; number of transducers: 256) and large image sizes (416×416 pixels). With DeepMB, clinical MSOT can provide high quality feedback during live imaging and thus facilitate advanced dynamic imaging applications. MSOT is a high-resolution functional imaging modality that can non-invasively quantify a broad range of pathophysiolog ical phenomena by accessing the endogenous contrast of chromophores in tissue. For details, see e.g. Taruttis, A. and V. Ntziachristos, Advances in real-time multi-spectral optoacoustic imaging and its applications. Nature Photonics, 2015. 9 (4): p. 219-227.

FIG. 1 shows a schematic of a preferred DeepMB pipeline for training, in vivo imaging and evaluation. (a) Real-world images, preferably obtained from a publicly available dataset, are used to generate synthetic sinograms by applying an accurate physical forward model of the scanner. SoS denotes the speed-of-sound. (b) In vivo sinograms are acquired from diverse anatomical locations in patients. (c) Optoacoustic images are reconstructed via iterative model-based reconstruction for the purpose of generating reference images for either the synthetic dataset (A) or the in vivo dataset (B). (d) Network training is conducted by using the synthetic data as sets for training and validation (C), while the in vivo data constitutes the test set (D). A domain transformation is first applied to the input sinograms via a delay operation to map the time samples values into the image space. The SoS is then one-hot encoded and concatenated as additional channels. A U-Net convolutional neural network is subsequently applied to the channel stack to regress the final image. The loss is calculated between the network output and the corresponding reference image (see below for further details about the network training).

Preferably, the framework can be applied to a modern handheld optoacoustic scanner, in particular an MSOT scanner, e.g. MSOT Acuity Echo (iThera Medical GmbH, Munich, Germany), with SoS values ranging from 1475 m/s to 1525 m/s in steps of 5 m/s.

Preferably, DeepMB is trained using input sinograms synthesized from general-feature images, also referred to as “initial images”, e.g. photographs of real-world or everyday situations, to facilitate the learning of an unbiased and universally applicable reconstruction operator. These sinograms are preferably generated by employing a diverse collection of publicly available real-world images as initial pressure distributions and simulating thereof the signals recorded by the acoustic transducers with an accurate physical model of the considered scanner (FIG. 1a).

In present example, the initial images are photographs from arbitrary objects and/or scenes taken by a conventional camera. Alternatively or additionally, the initial images can preferably be medical and/or in-vivo images obtained by a medical and/or tomographic imaging modality, for example a CT, MRT, ultrasound and/or optoacoustic imaging modality.

Preferably, the SoS values for the forward simulations are drawn uniformly at random from the considered range for each image. Ground-truth images for the synthesized sinograms are computed via model-based reconstruction (FIG. 1c).

FIG. 1d shows a preferred deep neural network architecture of DeepMB, which inputs a sinogram (either synthetic or in vivo) and a SoS value and outputs the final reconstructed image. The underlying design is preferably based on the U-net architecture augmented with two extensions that were found to be advantageous for the network to learn and express the effects of the different input SoS values onto the reconstructed images: (1) all signals are were mapped from the input sinogram to the image domain with a delay operation based on the given input SoS value and (2) the input SoS value (one-hot encoded and concatenated as additional channels) is passed to the trainable convolutional layers of the network. A detailed description of the network training is given below.

After training, the applicability of DeepMB to clinical data can be tested with a diverse dataset of in vivo sinograms acquired by scanning patients at different anatomical locations each (FIG. 1b). The corresponding ground-truth images of the acquired in vivo test sinograms can be obtained analogously to the training data via model-based reconstruction. Preferably, and inference time of less than 10 ms per sample can be achieved on a modern GPU (GeForce RTX 3090).

### Handheld MSOT Imaging System

Preferably, a handheld MSOT scanner system is used, which is equipped with a multi-wavelength laser that illuminates tissues with short laser pulses (<10 ns) at a repetition rate of 25 Hz. Preferably, the scanner features a custom-made ultrasound detector (IMASONIC SAS, Voray-sur-l'Ognon, France) with the following characteristics: Number of piezoelectric elements: 256; Concavity radius: 4 cm; Angular coverage: 125°; Central frequency: 4 MHz. Parasitic noise generated by light-transducer interference is reduced via optical shielding of the matching layer, yielding an extended 120% frequency bandwidth. The raw channel data for each optoacoustic scan is preferably recorded with a sampling frequency of 40 MHZ during 50.75 μs, yielding a sinogram of size 2030×256 samples. Co-registered B-mode ultrasound images can also be acquired and interleaved at approximately 6 Hz for live guidance and navigation. During imaging, optoacoustic backprojection images as well as B-mode ultrasound images can be displayed in real time on the scanner monitor for guidance.

### Acquisition of In Vivo Test Sinograms

To collect in vivo data for DeepMB evaluation six healthy volunteers were scanned. The involved participants were three females and three males, aged from 20 to 36 years (mean age: 28.3±5.7). Self-assessed skin color according to the Fitzpatrick scale was type II (2 participants), type III (3 p.), and type IV (1 p.). Self-assessed body type was ectomorph (2 p.), mesomorph (3 p.), and endomorph (1 p.).

For each participant, between 25 and 29 different combinations of anatomical locations and probe orientations were scanned: biceps, thyroid, carotid, calf (each left/right and transversal/longitudinal), elbow, neck, colon (each left/right), and breast (each left/right and top/bottom, female participants only). For each combination of anatomical location and probe orientation, we conducted between one and four acquisitions. During each acquisition, sinograms for approximately 10 s at wavelengths cyclically iterating from 700 to 980 nm in steps of 10 nm were recorded. Then, per acquisition, the 29 consecutively acquired sinograms were selected for which minimal motion in the interleaved ultrasound images was observed, amounting to a total of 4814 in vivo test sinograms.

Finally, all selected in vivo sinograms were band-pass filtered between 100 KHz and 12 MHz to remove frequency components beyond the transducer bandwidth and cropped the first 110 time samples to remove device-specific noise present at the beginning of the sinograms.

### Determination of the SoS Values

To evaluate DeepMB reconstructions under both in-focus and out-of-focus conditions, the SoS value of all in vivo test scans can be manually tuned. Preferably, a SoS step size of 5 m/s can be used to enable SoS adjustments slightly below the system spatial resolution (approximatively 200 μm). It was found that the range of optimal SoS values is approximately 1475-1525 m/s for the in vivo dataset, and therefore the same range can be used to define the supported input SoS values of the DeepMB network.

For each scan, the SoS value that resulted in the most well-focused reconstructed image was manually selected. To speed up tuning, the optimal SoS values was selected based on approximate and high-frequency-dominated reconstructions that was computed by applying the transpose model of the system to the recorded sinograms. Furthermore, the SoS was tuned only for scans at 800 nm and adopted the values for all scans at other wavelengths acquired at the time exploiting their spatial co-registration due to the absence of motion (see previous sections for details).

### Synthesis of Sinograms for Training and Validation

For network training and validation, optoacoustic sinograms are preferably synthesized with an accurate physical forward model of imaging process that incorporates the total impulse response of the system, parametrized by a SoS value drawn uniformly at random from the range (1475-1525 m/s) with step size 5 m/s. Real-world images serving as initial pressure distributions for the forward simulations can be randomly selected from the publicly available PASCAL Visual Object Classes Challenge 2012 (VOC2012) dataset, converted to mono-channel grayscale, and resized to 416×416 pixels. After the application of the forward model, each synthesized sinogram can be scaled by a factor drawn uniformly at random from the range (0-450) to match the dynamic range of in vivo sinograms.

### Image Reconstruction

To generate ground-truth optoacoustic images, all sinograms (synthetic as well as in vivo) were reconstructed via iterative-model-based. Preferably, Shearlet L¹ was used to tackle the ill-posedness of the inverse problem. Shearlet L¹ regularization is a convex relaxation of Shearlet sparsity, which can reduce limited-view artifacts in reconstructed images, because Shearlets provide a maximally-sparse approximation of a larger class of images (known as cartoon-like functions) with a provably optimal encoding rate, see e.g. Kutyniok, G. and W.-Q. Lim, Compactly supported shearlets are optimally sparse. Journal of Approximation Theory, 2011. 163(11): p. 1564-1589.

The optimal pressure field to find is characterized as

$${p}_{0} : = arg {min}_{p \geq 0} {‖ {M}_{SoS} p - s ‖}_{2} + λ {‖ SH ( p ) ‖}_{1} ,$$

where p₀ is the reconstructed image, M_{SoS} is the forward model of the imaging process for the selected reconstruction SoS, s is the input sinogram, λ is the regularization parameter tuned via an L-curve, SH is the Shearlet transform, and ∥ . . . ∥ₙ is the n-norm. The minimization problem is preferably solved via bound-constrained sparse reconstruction by separable approximation, see e.g. Wright, S. J., R. D. Nowak, and M. A. T. Figueiredo, Sparse Reconstruction by Separable Approximation. IEEE Transactions on Signal Processing, 2009. 57 (7): p. 2479-2493 and Chartrand, R. and B. Wohlberg. Total-variation regularization with bound constraints. in 2010 IEEE International Conference on Acoustics, Speech and Signal Processing. 2010. All images can be reconstructed, e.g., with a size of 416×416 pixels and a field of view of 4.16×4.16 cm². For comparison purposes, all images can also be reconstructed using the backprojection formula, see e.g. Kunyansky, L. A., Explicit inversion formulae for the spherical mean Radon transform. Inverse Problems, 2007. 23 (1): p. 373-383 and Kuchment, P. and L. Kunyansky, Mathematics of Photoacoustic and Thermoacoustic Tomography, in Handbook of Mathematical Methods in Imaging, O. Scherzer, Editor. 2011, Springer New York: New York, NY. p. 817-865.

### Network Training

DeepMB can be trained—either on synthetic or in vivo data—for example, for 300 epochs using stochastic gradient descent with batch size=4, learning rate=0.01, momentum=0.99, and per epoch learning rate decay factor=0.99. The network loss can be calculated as the mean square error between the output image and the reference image. The final model was selected based on the minimal loss on the validation dataset.

To facilitate training, all input sinograms can be scaled scaled by K=450⁻¹ to ensure that their values never exceed the range (−1 to 1). The same scaling factor can also be applied to all target images. Furthermore, the square root can be applied to all target reference images used during training and validation to reduce the network output values and limit the influence of high intensity pixels during loss calculation. When applying the trained network on in vivo test data, inferred images can be first squared and then scaled by K⁻¹, to revert the preprocessing operation.

When training on synthetic data to build the standard DeepMB model, for example 8000 sinograms can be used as train split and 2000 sinograms as validation split. An alternative scenario involving training on in vivo data to build the DeepMBinvivo models can be carried out as described hereafter: six different permutations can be conducted, with a 4/1/1 participants division between the train, validation, and test splits, respectively, each participant being once and only once part of the validation and test splits.

The DeepMB network is preferably based upon the U-Net architecture, preferably with a depth of 5 layers and a width of 64 features. The kernel and padding size are preferably (3, 3) and (1, 1), respectively. Preferably, biases are accounted for, and the final activation is the absolute value function.

### Data Residual Norm

To quantify the image fidelity of reconstructions from DeepMB, model-based, or backprojection, the data residual norm R can be evaluated, which is defined as

$$R := {‖ {M}_{S o S} {p}_{0} - s ‖}_{2}^{2} / {‖ s ‖}_{2}^{2} ,$$

where p₀ is the reconstructed image, M_{SoS} is the forward model from model-based reconstruction, s is the input sinogram, and ∥ . . . ∥₂ is the 2-norm. To enable a meaningful comparison between backprojection on one hand, versus non-negative model-based and DeepMB on the other hand, negative pixel values can be set to zero prior to residual calculation for backprojection images, to constrain the solution space to be analogous for all reconstruction methods. All images can be individually scaled using the linear degree of freedom in reconstructed optoacoustic image so that their data residual norms are minimal.

For evaluation of in-focus images, data residual norms can be calculated for model-based, DeepMB, and backprojection reconstructions with the optimal SoS values, for all 4814 samples from the in vivo test set. For evaluation of out-of-focus images, data residuals can be calculated for model-based and DeepMB reconstructions with all 11 SoS values, for a subset of 638 randomly selected in vivo samples.

FIG. 2 shows representative examples of the in vivo test dataset for different anatomical locations (abdomen: a-d, calf: e-h, biceps: i-l, carotid artery: m-p). The two leftmost columns correspond to deep model-based (DeepMB) and model-based (MB) reconstructions. The third column represents the absolute difference between DeepMB and model-based images. The rightmost column depicts the backprojection (BP) reconstructions. The shown field of view is 4.16×2.80 cm², each enlarged region is 0.41×0.41 cm².

FIG. 3 shows data residual norms of optoacoustic images from deep model-based (DeepMB), model-based (MB), and backprojection (BP) reconstruction. (a) Data residual norms of in-focus images reconstructed with optimal speed of sound (SoS) values, on all 4814 samples from the in vivo test set. (b) Data residual norms of out-of-focus images reconstructed with sub-optimal SoS values, on a subset of 638 samples. The five sub-panels depict the effect of SoS mismatch via gradual increase of the offset ΔSoS in steps of 10 m/s.

### Qualitative Evaluation

To evaluate the capacity of DeepMB to reconstruct high-quality images, all DeepMB images from the clinical dataset (FIG. 1b) are compared to their corresponding model-based reference images (FIG. 1c). FIG. 2 illustrates that the DeepMB reconstructions (FIG. 2 a, e, i, m) were systematically nearly-indistinguishable from the model-based references (FIG. 2 b, f, j, n), with no noticeable failures, outliers, or artifacts for any of the participants, anatomies, probe orientations, SoS values, or laser wavelengths. The similarity between the DeepMB and model-based images is also confirmed by their negligible pixel-wise absolute differences (FIG. 2 c, g, k, o). The zoomed region G in FIG. 2 i-k depicts one of the largest observed discrepancies between the DeepMB and model-based reconstructions, which manifests as minor blurring, showing that the DeepMB image is only marginally affected by these errors. In comparison, backprojection images (FIG. 2 d, h, l, p) exhibit notable differences from the reference model-based images and suffer from reduced spatial resolution and physically-nonsensical negative initial pressure values.

### Quantitative Evaluation

To quantify the image fidelity of DeepMB reconstructions, the data residual norm was calculated for all in vivo test images. The data residual norm measures the fidelity of a reconstructed image by computing the mismatch between the image and the corresponding recorded acoustic signals with regard to the accurate physical forward model of the used scanner, and is provably minimal for model-based reconstruction. The data residual norm can also be calculated for all model-based and backprojection reconstructions, for comparison purposes.

First, the calculated data residual norms of in-focus images reconstructed with optimal SoS values can be assessed to evaluate the fidelity of DeepMB images with the best possible quality (FIG. 3a). Data residual norms of DeepMB images (mean=0.156) are almost as low as the provably minimal data residual norms of model-based (MB) images (mean=0.139), whereas the data residual norms of backprojection (BP) images (mean=0.369) are substantially higher. The close agreement between data residual norms of DeepMB and model-based images confirms that both reconstruction approaches afford equivalent image qualities. In contrast, the data residual norms of backprojection images are markedly higher, which reaffirms the shortcomings of backprojection to accurately model the imaging process, and explains the lower image quality observed in FIG. 2 d, h, l, p.

Second, the data residual norms of out-of-focus images reconstructed with sub-optimal SoS values can be assessed to evaluate the fidelity of DeepMB images during imaging applications with a priori unknown SoS (FIG. 3b). Data residual norms of DeepMB images remain close to those of model-based images for all considered levels of mismatch between the optimal and the employed SoS, thus confirming that DeepMB and model-based images are similarly trustworthy independent of the selected SoS.

### Advantages of Synthesized Training Data

Finally, to assess the advantage of using synthesized training data for DeepMB to learn an accurate and general reconstruction operator, alternative DeepMB models can be trained on in vivo data instead of synthetic data. These models, referred to as DeepMBinvivo, inferred images with similar data residual norms (mean=0.155) as the standard DeepMB model. However, DeepMBinvivo images may contain visible artifacts, either at the left or right image borders, or in regions showing strong absorption at the skin surface. On the other hand, no artifacts are observed with the preferred training strategy of DeepMB (using synthesized training data), even when reducing the size of the synthetic training set from 8000 to 3500 to match the reduced amount of available in vivo training data.

As demonstrated above, DeepMB achieves three seminal features compared to previous approaches: Accurate generalization to in vivo measurements after training on synthesized sinograms that were derived from real-world images, dynamic adjustment of the reconstruction SoS during imaging, and compatibility with the data rates and image sizes of modern MSOT scanners. DeepMB therefore enables dynamic-imaging applications of optoacoustic tomography and deliver high-quality images to clinicians in real-time during examinations, furthering the clinical translation of this technology and leading to more accurate diagnoses and surgical guidance.

DeepMB is preferably trained on synthesized sinograms from real-world images, instead of in vivo images, because these synthesized sinograms afford a large training dataset with a versatile set of image features, allowing DeepMB to accurately reconstruct images with diverse features. In particular, such general-feature training datasets reduce the risk of encountering out-of-distribution samples (test data with features that are not contained in the training dataset) when applying the trained model to in vivo scans. In contrast, training a model on in vivo scans may systematically introduce the risk of overfitting to specific characteristics of the training samples and can lead to decreased image quality for never-seen-before scans that may involve different anatomical views, body types, skin colors, or disease states. It can be observed that alternative DeepMB in vivo models trained on in vivo data fail to adequately generalize to some in vivo test scans and introduce artifacts within the reconstructed images. Furthermore, using synthesized data instead of in vivo data alleviates the training of new DeepMB models because it obviates the need for recruiting and scanning a cohort of participants. Instead, training data can be automatically generated and used to straightforwardly obtain specifically-trained DeepMB models for new scanners or different reconstruction parameters.

Accurate generalization from synthesized training to in vivo test data is possible with DeepMB because the underlying inverse problem to solve (that is, regularized model-based reconstruction) is well-posed; for each input sinogram there is a unique and stable solution (i.e., the reconstructed image). Therefore, the network can learn a data transform that is agnostic to specific characteristics of the ground-truth images during training and generalizes to images with any content (be it synthesized or in vivo). In contrast, previous deep-learning-based optoacoustic reconstruction approaches were limited in their ability to generalize from synthesized training data to in vivo test data because the underlying inverse problems were ill-posed. More precisely, the targets used during the training of these deep neural networks were the true synthetic initial pressure images (left side in FIG. 1a), containing image information not available in the input sinogram due to limited angle acquisition, measurement noise, or finite transducer bandwidth. To restore the missing information, these deep neural network models were required to incorporate information from the training data manifold, which limited their generalization and provides a likely explanation for the rudimentary image quality previously reported for in vivo measurements.

The disclosed DeepMB methodology to increase the speed of iterative model-based reconstruction is also applicable to other optoacoustic reconstruction approaches. For instance, frequency-band model-based reconstruction or Bayesian optoacoustic reconstruction can disentangle structures of different physical scales and quantifying reconstruction uncertainty, respectively, but their long reconstruction times currently hinder their use in real-time applications. The methodology of DeepMB could also be exploited to accelerate parametrized (iterative) inversion approaches for other imaging modalities, such as ultrasound, X-ray computed tomography, magnetic resonance imaging, or, more generally, for any parametric partial differential equation. In conclusion, DeepMB is a fully operational software-based prototype for real-time model-based optoacoustic image reconstruction, which can be e.g. embedded into the hardware of a modern MSOT scanner to use DeepMB for real-time imaging in clinical applications.

### DeepOPUS

As already mentioned, the above explanations regarding the DeepMB implementation apply accordingly to the DeepOPUS implementation.

Preferably, DeepOPUS relates to a deep learning solution for simultaneous or joint reconstruction of co-registered optoacoustic (OA) and ultrasonic (US) images, so that synergistic effects between these two imaging modalities can be used, i.e. US image reconstruction is improved by considering information from OA data and/or OA image(s) and OA image reconstruction is improved by considering information from US data and/or US image(s). This will be explained in more detail in the following.

FIG. 4 shows a schematic representation to illustrate an improved US image reconstruction of reflective interfaces. As illustrated in FIG. 4a, a reflector within a sample cannot be detected by reflection ultrasound tomography, since signals are not reflected back to the transducer array, which acts both as a transmitter of ultrasound waves and a detector for reflected ultrasound waves. As illustrated in FIG. 4b, an optoacoustic source within the sample acts like a probe (emitting ultrasound waves) for the reflector, enabling tomographic reconstruction.

As a result, DeepOPUS allows for an improved reconstruction of [symbol] via the mapping (US data, OA data, p₀, SoS) [symbol] ┌, wherein p₀ acts as additional probe from within the sample, as illustrated in FIG. 4. In other words, knowing the pressure waves that are generated by the optoacoustic sources (given by p₀), one can recover additional information about the location of reflective interfaces from the acquired (and potentially reflected) OA signals for an improved reconstruction of ultrasonic images.

FIG. 5 shows a schematic representation to illustrate an improved OA image reconstruction (initial pressure distribution). As illustrated in FIG. 5a, detector arrays with limited angular coverage cannot detect optoacoustic sources that are elongated perpendicular to the array, since the generated waves are directional and do not hit the detector. As illustrated in FIG. 4b, knowledge of reflectors that reflect signals generated by elongated structures towards the detector allows detection of such structures.

As a result, DeepOPUS allows for an improved reconstruction of p₀ via the mapping (US data, OA data, SoS, ┌) [symbol] p₀, wherein reflections are removed via knowledge of ┌, limited angular coverage is reduced by signals reflected back to transducers via knowledge of ┌, as illustrated in FIG. 5. In other words: Optoacoustic waves that would normally not arrive at the detector array due to the limited angular coverage can potentially be reflected to the detectors at reflective interfaces (as given by ┌). Acquisition of such signals in combination with knowledge of the location of reflective interfaces allows to improve the reconstruction and mitigate limited view artefacts.

FIG. 6 shows a schematic representation to illustrate an improved reconstruction of the speed of sound distribution. As illustrated in FIG. 6a, reflection ultrasound data is not suitable for reconstruction of the speed of sound distributions due to its limited information about times of flight through tissue layers. As illustrated in FIG. 6b, optoacoustic sources can serve as probes for transmission ultrasound data that simplify speed of sound reconstruction significantly.

As a result, DeepOPUS allows for an improved reconstruction of SoS in reflection mode US with the help of OA via the mapping (US data, OA data, ┌, p₀) [symbol] SoS, wherein OA data is partial transmission data. In other words: Although reconstruction of the speed of sound distribution from reflective US data (i.e., waves that are at least once reflected within the sample) alone is highly ill-posed and, therefore, only achievable with rudimentary quality, knowledge of the locations of optoacoustic sources (as given by p₀) that send acoustic waves through the tissue towards the detector without being reflected provides transmission data from which the reconstruction of the speed of sound distribution is possible with much higher quality and reliability.

In the following, preferred aspects of present disclosure are illustrated with reference to FIGS. 7 and 8, which relate to the DeepOPUS implementation. Because the DeepMB implementation is a special case of the DeepOPUS implementation, the following explanations apply accordingly to the DeepMB implementation.

FIG. 7 shows an exemplary schematic to illustrate aspects of present disclosure, in particular a method for training an artificial neural network 1 for reconstructing optoacoustic and ultrasonic images from signals generated by an imaging apparatus 2 for optoacoustic and ultrasonic imaging.

The method for training the artificial neural network 1 uses a model M_{c} of the imaging apparatus 2, wherein the model M_{c} characterizes a relation between i) a spatial distribution of acoustic sources 3 (for simplification, only three acoustic sources are shown) contained in an object 3′ emitting and/or reflecting acoustic waves 4 in response to irradiating the object 3′ with electromagnetic radiation 6a and ultrasonic waves 6b generated by an irradiation device 6 of the imaging apparatus 2, and ii) signals s_{OA}, s_{US} generated by detection elements 5 of the imaging apparatus 2 upon detecting the acoustic waves 4.

Several training signal sets s_{OA}, s_{US} are provided, wherein each training signal set s_{OA}, s_{US} comprises a plurality of training signals which were generated by the imaging apparatus 2 upon imaging objects (i.e. by irradiating objects with electromagnetic radiation 6a and acoustic waves 6b and detecting the generated optoacoustic and ultrasonic waves 4 with the detection elements 5). Alternatively or additionally, the training signal sets s_{OA}, s_{US} can be obtained by simulating an imaging of objects by the imaging apparatus 2 based on the model M_{c} of the imaging apparatus 2.

Further, training image data sets X*_{OA}, x*_{US}, c* are reconstructed, by means of a model-based reconstruction based on the model M_{c} of the imaging apparatus 2, from the training signal sets s_{OA}, s_{US}, wherein each training image data set x*_{OA}, x*_{US}, c* comprises image data x*_{OA}, x*_{US} relating to an optoacoustic and ultrasonic image of an object, and optionally a speed of sound distribution c*.

As illustrated in FIG. 7, the speed of sound distribution c* can be reconstructed from the training signal sets s_{OA}, s_{US}. Alternatively, as indicated by brackets [ . . . ], the speed of sound distribution c can be determined a-priori and given as an input to the model-based reconstruction and the artificial neural network 1.

The artificial neural network 1 comprises an input layer 1a and an output layer 1b and is trained by i) inputting the training signal sets Son, s_{US} at the input layer 1a, ii) obtaining, for each inputted training signal set s_{OA}, s_{US}, an output image data set x_{OA}, x_{US}, and optionally a speed of sound distribution c, which is outputted at the output layer 1b, and iii) comparing each output image data set x_{OA}, x_{US}, c with the training image data set X*_{OA}, x*_{US}, c* which was reconstructed from the respectively inputted training signal set s_{OA}, s_{US}.

FIG. 8 shows an exemplary schematic to illustrate aspects of present disclosure, in particular a method and corresponding system for optoacoustic and ultrasonic imaging.

The imaging apparatus 2 comprises an irradiation device 6 configured to irradiate, subsequently or simultaneously, an object 3′ comprising a plurality of acoustic sources 3 with electromagnetic radiation 6a and acoustic waves 6b. For simplification, only three acoustic sources 3 are shown. Detection elements 5 of the imaging apparatus 2 are configured to generate a set of signals s_{OA}, s_{US} by detecting acoustic waves 4 emitted or reflected, respectively, by the object 3′ (i.e. by the acoustic sources 3 of the object 3′) in response to irradiating the object 3′ with the electromagnetic radiation 6a and acoustic waves 6b, respectively.

A processor 7 is configured to reconstruct an optoacoustic and ultrasonic image x_{OA}, x_{US} of the object 3′ from the set of signals s_{OA}, s_{US} by inputting the set of signals s_{OA}, s_{US} at the input layer 1a of the trained artificial neural network 1, and obtaining at least one optoacoustic and ultrasonic image x_{OA}, x_{US}, and optionally a speed of sound distribution c, which is outputted at the output layer 1b of the trained artificial neural network 1.

As illustrated in FIG. 8, the speed of sound distribution c can be obtained from the training signal sets s_{OA}, s_{US} inputted into the artificial neural network 1. Alternatively, as indicated by brackets [ . . . ], the speed of sound distribution c can be determined a-priori and given as an input to the artificial neural network 1.

1. A method for training an artificial neural network for reconstructing optoacoustic and ultrasonic images from signals generated by an imaging apparatus for optoacoustic and ultrasonic imaging, the method comprising:
a) providing a model of the imaging apparatus, the model characterizing a relation between i) a spatial distribution of acoustic sources emitting and reflecting acoustic waves and ii) signals generated by detection elements of the imaging apparatus upon detecting the acoustic waves,
b) providing several training signal sets, each training signal set comprising a plurality of optoacoustic and ultrasonic training signals which were i) generated by the imaging apparatus upon imaging objects and/or ii) obtained by simulating an imaging of objects by the imaging apparatus based on the model of the imaging apparatus,
c) reconstructing, based on the model of the imaging apparatus, several training image data sets from the training signal sets, each training image data set comprising image data relating to an optoacoustic and an ultrasonic image of an object, and
d) training the artificial neural network, which comprises an input layer and an output layer, the training comprising i) inputting the training signal sets at the input layer, ii) obtaining, for each inputted training signal set, an output image data set which is outputted at the output layer, and iii) comparing each output image data set with the training image data set which was reconstructed from the respectively inputted training signal set.

2. The method according to claim 1, the model characterizing at least one of the following: i) a propagation of the acoustic waves from the acoustic sources towards the detection elements, ii) a response of the detection elements upon detecting the acoustic waves, and/or iii) a noise of the imaging apparatus.

3. The method according to claim 2, wherein characterizing the propagation of the acoustic waves includes at least one of the following: i) an acoustic wave propagation model, which is the same for the propagation of both emitted optoacoustic waves and reflected ultrasound waves, ii) a propagation of the acoustic waves through a medium with an inhomogeneous speed of sound distribution, and/or iii) a reflection of the acoustic waves at one or more reflective interfaces in the medium.

4. The method according to claim 1, wherein at least some of the training signal sets comprise training signals which were obtained, in particular synthesized, by simulating imaging of objects by the imaging apparatus based on i) the model of the imaging apparatus and ii) initial images of objects which were obtained by any imaging apparatus.

5. The method according to claim 1, wherein each training signal set comprises a plurality of optoacoustic training signals and a plurality of ultrasonic training signals, and wherein reconstructing at least one training image data set from at least one training signal set is based on a simultaneous and/or joint consideration of the respective optoacoustic training signals and ultrasonic training signals comprised in the at least one training signal set.

6. The method according to claim 1, wherein reconstructing at least one training image data set from at least one training signal set comprises:
i) calculating, based on the model of the imaging apparatus taking into account a propagation of the acoustic waves through a medium with a, in particular pre-defined or reconstructed, speed of sound distribution, several prediction signal sets from several varying image data sets,
ii) calculating, for each of the varying image data sets, a second distance metric between the respective prediction signal set and the training signal set, and
iii) determining at least one image data set for which the second distance metric between the respective prediction signal set and the at least one training signal set exhibits a minimum, wherein the at least one training image data set is the at least one determined image data set.

7. The method according to claim 6, wherein comparing the output image data set with the respective training image data set comprises determining a loss function which is given by:
a third distance metric, in particular a means squared error, between the output image data set, on the one hand, and the respective training image data set and speed of sound distribution reconstructed from the respective training signal set, on the other hand, and/or
the first and/or second distance metric which is applied to the output image data set.

8. The method according to claim 1, wherein the at least one artificial neural network is given by i) a single deep neural network or ii) a cascade of multiple deep neural networks.

9. The method according to claim 1, wherein the training comprises (one-step process) i) inputting the training signal sets at the input layer, ii) obtaining, for each inputted training signal set, both the output image data set and an output speed of sound distribution which are outputted at the output layer, and iii) comparing each output image data set and output speed of sound distribution (c) with the training image data set and, respectively, a training speed of sound distribution which were reconstructed from the respectively inputted training signal set.

10. The method according to claim 1, wherein the training comprises (two-step process),
i) inputting the training signal sets at the input layer, ii) obtaining, for each inputted training signal set, an output speed of sound distribution which is outputted at the output layer, and iii) comparing each output speed of sound distribution with a training speed of sound distribution which was reconstructed from the respectively inputted training signal set, and subsequently
i) inputting the training signal sets and the output speed of sound distribution at the input layer, ii) obtaining, for each inputted training signal set and output speed of sound distribution, the output image data set which is outputted at the output layer, and iii) comparing each output image data set with the training image data set which was reconstructed from the respectively inputted training signal set.

11. A method for reconstructing an optoacoustic and ultrasonic image from a set of signals generated by an imaging apparatus for optoacoustic and ultrasonic imaging and comprising a plurality of optoacoustic signals and a plurality of ultrasonic signals, the method comprising:
inputting the set of signals at an input layer of the artificial neural network which has been trained by the method according to claim 1, and
obtaining at least one optoacoustic and ultrasonic image which is outputted at an output layer of the trained artificial neural network.

12. The method according to claim 11, wherein the optoacoustic signals and ultrasonic signals comprised by the set of signals are simultaneously and/or jointly inputted at the input layer of the trained artificial neural network, and/or the optoacoustic image and ultrasonic image are simultaneously and/or jointly outputted at the output layer of the trained artificial neural network.

13. A method for optoacoustic and ultrasonic imaging comprising:
irradiating an object with electromagnetic radiation and acoustic waves and generating a set of signals by detecting acoustic waves emitted or reflected, respectively, by the object in response thereto by means of an imaging apparatus for optoacoustic and ultrasonic imaging, the set of signals comprising a plurality of optoacoustic signals and a plurality of ultrasonic signals, and
reconstructing an optoacoustic and ultrasonic image of the object from the set of signals by the method according to claim 11.

14. A system for optoacoustic and ultrasonic imaging comprising:
an imaging apparatus for optoacoustic and ultrasonic imaging, the imaging apparatus comprising an irradiation device configured to irradiate an object with electromagnetic radiation and acoustic waves, and a detection device configured to generate a set of signals by detecting acoustic waves emitted or reflected, respectively, by the object in response to irradiating the object with the electromagnetic and acoustic waves, the set of signals comprising a plurality of optoacoustic signals and a plurality of ultrasonic signals, and
a processor configured to reconstruct an optoacoustic and ultrasonic image of the object from the set of signals by the method according to claim 11.

15. A computer program product causing a computer, computer system and/or distributed computing environment to execute the method according to claim 1.

16. A computer program product comprising instructions causing a processor to execute the steps of the method according to claim 11.
